(19) United States
(12) Patent Application Publication
(10) Pub. No.: US 2012/0172593 A1
(43) Pub. Date: Jul. 5, 2012

(54) META-STABLE STATE NITROGEN-CONTAINING POLYMER

(75) Inventors: Li-Duan Tsai, Hsinchu City (TW); Yueh-Wei Lin, Hsinchu City (TW); Jason Fang, Taipei City (TW); Cheng-Liang Cheng, Hsinchu City (TW); Jing-Pin Pan, Hsinchu County (TW); Tsung-Hsiung Wang, Taichung City (TW)

(73) Assignee: INDUSTRIAL TECHNOLOGY RESEARCH INSTITUTE, Hsinchu (TW)

(21) Appl. No.: 13/109,008

(22) Filed: May 17, 2011

(30) Foreign Application Priority Data
Dec. 29, 2010 (TW) 99146605

(51) Int. Cl. C07D 401/14; C07D 403/14

(52) U.S. Cl. 544/198; 548/313.1; 544/238; 546/256

(57) ABSTRACT

A meta-stable state nitrogen-containing polymer formed by reacting Compound (A) and Compound (B) is described. Compound (A) is a monomer having a reactive terminal functional group. Compound (B) is a heterocyclic amino aromatic derivative as an initiator. The molar ratio of Compound (A) to Compound (B) is from 10:1 to 1:10. The meta-stable state nitrogen-containing polymer has a variance less than 2% in its narrow molecular weight distribution after being retained at 55° C. for one month.

### CROSS-REFERENCE TO RELATED APPLICATION

This application claims the priority benefit of Taiwan application serial no. 99146605, filed Dec. 29, 2010. The entirety of the above-mentioned patent application is hereby incorporated by reference herein and made a part of this specification.

### BACKGROUND

1. Technical Field

This application relates to a polymer, and more generally to a meta-stable state nitrogen-containing polymer.

2. Description of Related Art

Polymeric materials are inseparable from human daily life. For example, blend fabric clothes, plastic bags, automobile tires and bumpers, precise electric materials or even artificial bones are associated with polymeric materials. A polymer is usually formed from monomers through a polymerization reaction. Due to the time-consuming polymerization process, “two-liquid type” polymer and “one-liquid type” polymer are developed to shorten the synthesis time and broaden the application.

The “two-liquid type” polymer includes a main agent and an auxiliary agent. These two agents are mixed before using. For example, the epoxy resin adhesive of two-liquid type includes a resin as a main agent and a hardener as an auxiliary agent. Due to separate preservation of the agents, the “two-liquid type” polymer can be preserved for a long period of time, without interaction between the agents that causes the polymer degradation. Further, the mixture of the agents usually has better quality (such as heat resistance) as compared with the “one-liquid type” polymer. However, an additional mixing step before using is required.

The “one-liquid type” polymer is formed by mixing all required materials. For example, the epoxy resin adhesive of one-liquid type includes a resin, a solvent, a hardener and an inhibitor, etc. The “one-liquid type” polymer can be used right after unsealing. However, the preservation is difficult. Usually, it is required to be preserved under low temperature (e.g. below room temperature) to avoid the polymer degradation.

Both “two-liquid type” polymer and “one-liquid type” polymer have the problem in which the viscosity of the polymer is increased as it is exposed to air too long after unsealing. As a result, the polymer is hardened and can not be used anymore. Therefore, a material with the above advantages but without the above drawbacks is deeply desired so as to broaden the application.

### SUMMARY

Accordingly, the disclosure provides a meta-stable state nitrogen-containing polymer, in which a mixing step is omitted, long preservation is possible at room temperature (or above room temperature), and abrupt change in viscosity after unsealing is avoided.

A meta-stable state nitrogen-containing polymer is introduced herein. The meta-stable state nitrogen-containing polymer formed by reacting Compound (A) and Compound (B). Compound (A) is a monomer with a reactive terminal functional group. Compound (B) is a heterocyclic amino aromatic derivative as an initiator. A molar ratio of Compound (A) to Compound (B) is from 10:1 to 1:10.

Based on the above, the meta-stable state nitrogen-containing polymer of the disclosure can be stored at low or middle temperature for a long period of time, while maintaining its stable properties such as viscosity and particle size distribution. Furthermore, the meta-stable state nitrogen-containing polymer has part of the functional groups remained, and the unreacted function groups can be re-induced to react by applying an appropriate temperature or voltage; and thus, the purpose of the application can be easily achieved.

In order to make the features and advantages of the application clearer and more understandable, the following embodiments are illustrated in detail with reference to the appended drawings.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIGS. 1-9 are GPC diagrams of meta-stable state nitrogen-containing polymers of Examples 1-9 according to the disclosure;

FIG. 10 is a diagram illustrating the variation of GPC of the meta-stable state nitrogen-containing polymer of Example 3 according to the disclosure over time; and

FIG. 11 is a diagram illustrating the variation of viscosity of the meta-stable state nitrogen-containing polymer of Example 3 according to the disclosure over time.

### DESCRIPTION OF EMBODIMENTS

A meta-stable state nitrogen-containing polymer of the disclosure is formed by reacting Compound (A) and Compound (B). Compound (A) is a monomer with a reactive terminal functional group. Compound (B) is a heterocyclic amino aromatic derivative as an initiator. A molar ratio of Compound (A) to Compound (B) is from 10:1 to 1:10

Compound (B) is represented by one of Formula (1) to Formula (9):

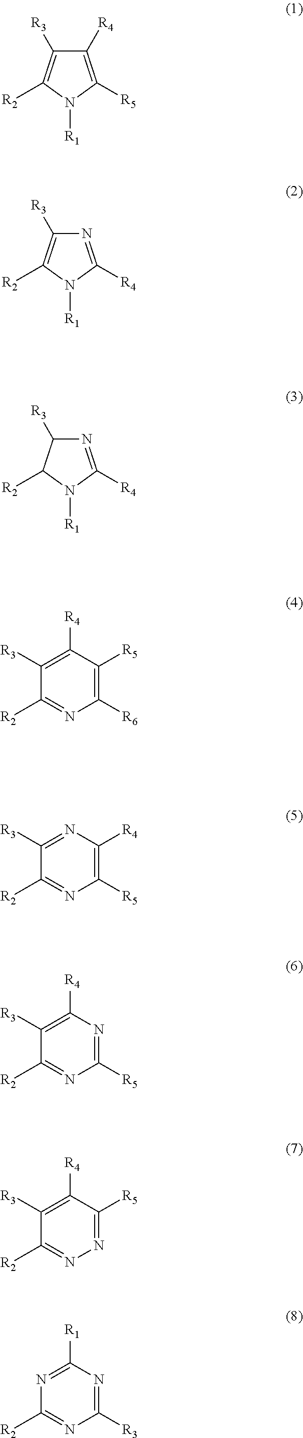

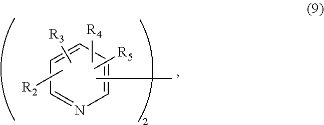

wherein R₁ is hydrogen, alkyl, alkenyl, phenyl, dimethylamino, or —NH₂; and R₂, R₃, R₄ and R₅ are each independently hydrogen, alkyl, alkenyl, halo, or —NH₂.

In an embodiment, examples of Compound (B) are as shown in Table 1.

TABLE 1

| Chemical Name | Structural Formula |
|---|---|
| Imidazole | 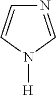 |
| Pyrrole | 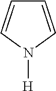 |
| Pyridine | 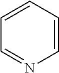 |
| 4-tert-butylpyridine | 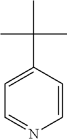 |
| 3-butylpyridine | 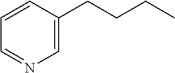 |
| 4-dimethylaminopyridine | 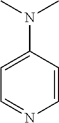 |
| 2,4,6-triamino-1,3,5,-triazine (melamine) | 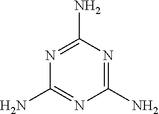 |
| 2,4-dimethyl-2-imidazoline | 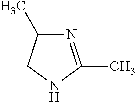 |
| Pyridazine | 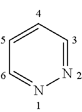 |
| Pyrimidine | 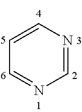 |
| Pyradine | 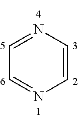 |

In another embodiment, Compound (B) may also be an imidazole derivative or a pyrrole derivative.

In an embodiment, Compound (A) is a maleimide monomer, represented by one of Formula (10) to Formula (13):

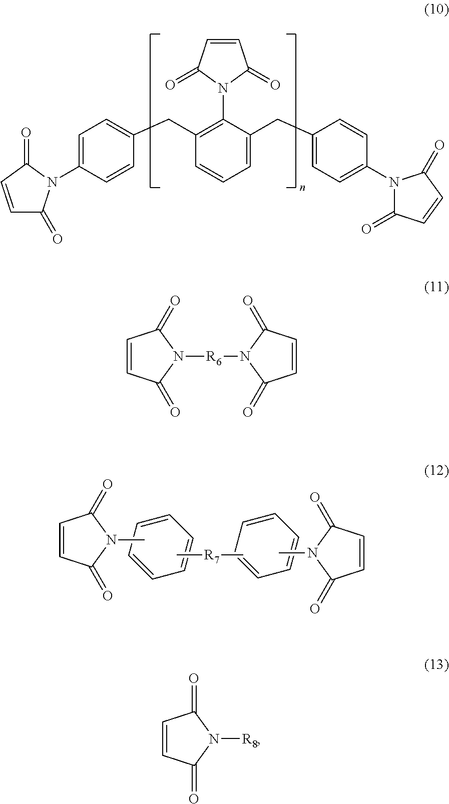

wherein n is an integer of 0 to 4; R₆ is —RCH₂R′—, —RNH₂R—, —C(O)CH₂—, —R′OR″OR′—, —CH₂OCH₂—, —C(O)—, —O—, —O—O—, —S—, —S—S—, —S(O)—, —CH₂S(O)CH₂—, —(O)S(O)—, —C₆H₅—, —CH₂(C₆H₅)CH₂—, —CH₂(C₆H₅)(O)—, phenylene, biphenylene, substituted phenylene, or substituted biphenylene, R is hydrogen or C₁₋₄ alkyl, R′ is C₁₋₄ alkyl, and R″ is C₁₋₄ alkyl; R₇ is —RCH₂—, —C(CH₃)₂—, —O—, —O—O—, —S—, —S—S—, —(O)S(O)—, or —S(O)—; and R₈ is hydrogen, C₁₋₄ alkyl, phenyl, benzyl, cyclohexyl, or N-methoxy carbonyl.

Examples of the maleimide monomer are as shown in Table 2.

TABLE 2

| Chemical Name | Structural Formula |
|---|---|
| 4,4′-diphenylmethane bismaleimide | 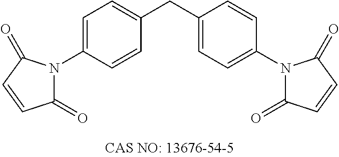 |
| Oligomer of phenylmethane maleimide | 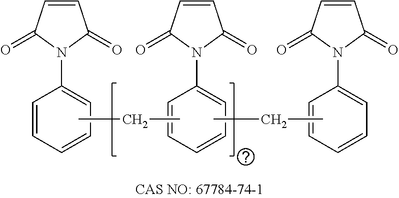 |
| m-phenylene bismaleimide | 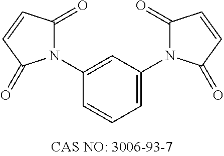 |
| 2,2′-bis[4-(4- maleimidophenoxy) phenyl]propane | 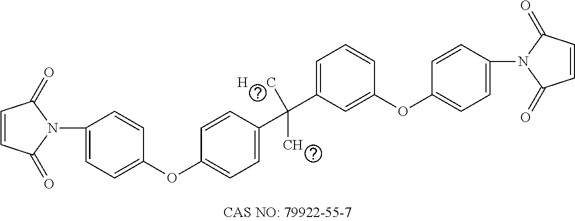 |
| 3,3′-dimethyl- 5,5′-diethyl-4,4′- diphenylmethane bismaleimide | 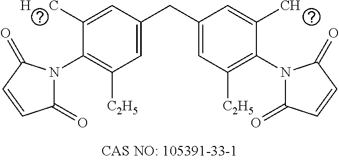 |
| 4-methyl-1,3-phenylene bismaleimide | 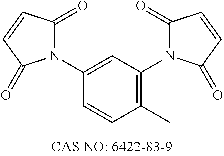 |
| 1,6′-bismaleimide- (2,2,4-trimethyl)hexane | 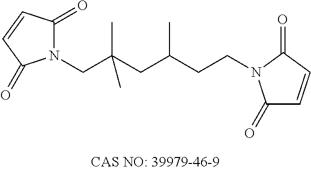 |
| 4,4′-diphenylether bismaleimide | 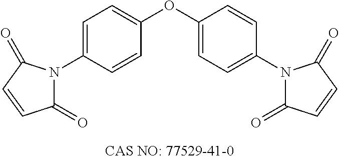 |
| 4,4′-diphenylsulfone bismaleimide | 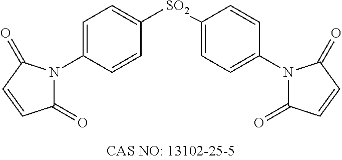 |
| 1,3-bis(3- maleimidophenoxy)benzene | 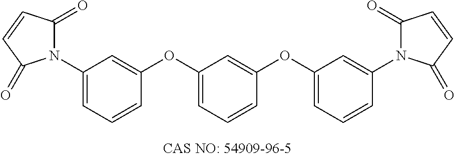 |
| 1,3-bis(4- maleimidophenoxy)benzene | 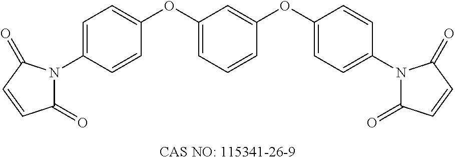 |

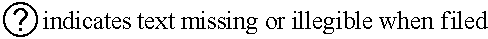

In another embodiment, Compound (A) may also be poly(ethylene glycol) dimethacrylate, bis[[4-[(vinyloxy)methyl]cyclohexyl]methyl]isophthalate, or triallyl trimellitate.

Next, a synthesis method of the meta-stable state nitrogen-containing polymer of the disclosure is described. Firstly, Compound (A) is dissolved in a solvent, to form a mixture solution. Then, Compound (B) is added into the mixture solution in batches, and thermally polymerized by heating. The molar ratio of Compound (A) to Compound (B) is, for example, from 10:1 to 1:10, or from 1:1 to 5:1.

The solvent includes y-butyrolactone (GBL), ethylene carbonate (EC), propylene carbonate (PC), N-methyl pyrollidone (NMP), and other high-polarity solvents, and is capable of providing high dissolution ability, which is beneficial to the thermal polymerization of the reactants. Moreover, the application scope of the mixture solution is widened by the flexible variation of the solid content.

Compound (B) may be added in 2-30 equivalent batches or non-equivalent batches, or in 4-16 batches; an adding time interval may be 5 minutes to 6 hours, and preferably 15 minutes to 2 hours; and the reaction may be performed at a temperature of 60-150° C., or 120-140° C. Furthermore, reaction time refers to a time that the reaction lasts after Compound (B) is completely added, and may be 0.5 hour to 48 hours, or 1 hour to 24 hours.

That is to say, Compound (B) is gradually added, in batches at a time interval (multiple times, e.g. twice or more times), into the mixture solution of Compound (A)/solvent system at the reaction temperature for thermal polymerization, so that gelation or a network structure generated by over reaction caused by adding of Compound (B) completely at one time can be avoided. The meta-stable state nitrogen-containing polymer synthesized in the disclosure

can be stored at room temperature (or higher) for a long time, and the viscosity thereof will not change drastically after unsealing. Furthermore, the meta-stable state nitrogen-containing polymer of the disclosure has part of the reactive functional groups remained, thus being beneficial to the subsequent processing, and optionally, the unreacted functional groups may be facilitated to react by heating or applying a voltage. In an embodiment, the meta-stable state nitrogen-containing polymer is re-induced to react at a temperature of 160-200° C., to convert the monomer into the polymer completely.

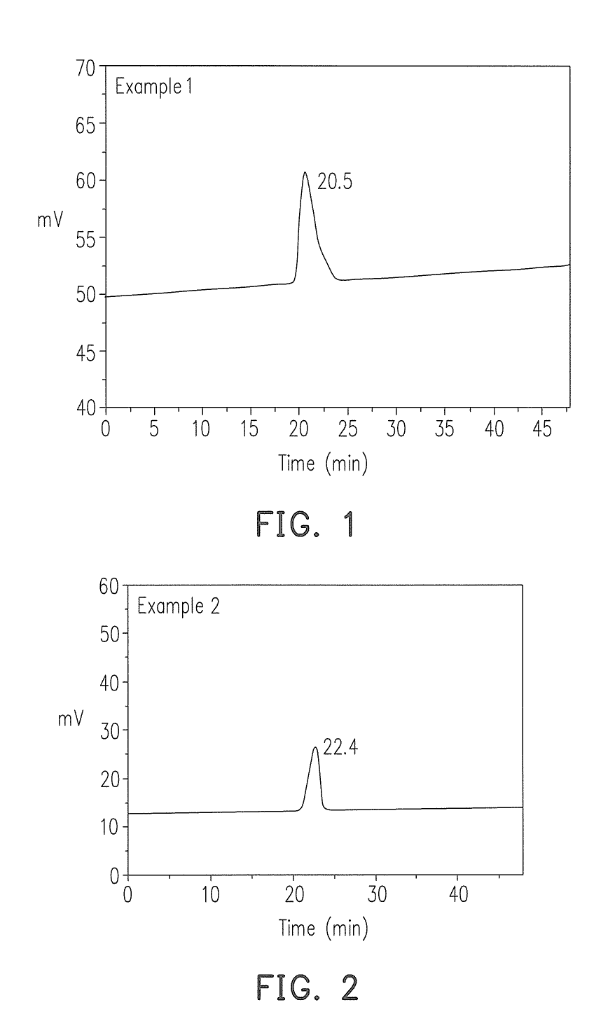

Hereinafter, multiple synthesis examples are illustrated to verify the efficacy of the disclosure. FIGS. 1-9 are gel permeation chromatograms (GPCs) of meta-stable state nitrogen-containing polymers of Examples 1-9 according to the disclosure, in which the longitudinal axis is in minivolt (mV), and refers to signal strength (or sensitivity) of a detector, and the horizontal axis is in time.

### EXAMPLE 1

Firstly, oligomer of phenylmethane maleimide (Compound (A)) was dissolved in EC/PC in an amount of 3%, to form a mixture solution. Next, 2,4-dimethyl-2-imidazoline (Compound (B)) was added into the mixture solution in batches, for thermal polymerization at 130° C. for 8 hours, so as to obtain a meta-stable state nitrogen-containing polymer of Example 1. The molar ratio of 3% oligomer of phenylmethane maleimide to 2,4-dimethyl-2-imidazoline was 2:1.

The meta-stable state nitrogen-containing polymer of Example 1 was a narrow polydispersity polymer having a gel permeation chromatography (GPC) peak time of 20.5 min, as shown in FIG. 1. Furthermore, the meta-stable state nitrogen-containing polymer of Example 1 was re-induced to react at a temperature of 186° C., to convert the monomer into the polymer completely. Polydispersity index (PDI) is defined as weight average molecular weight divided by number average molecular weight.

### EXAMPLE 2

Firstly, 4,4′-diphenylmethane bismaleimide (Compound (A)) was dissolved in GBL in an amount of 5%, to form a mixture solution. Next, 2,4-dimethyl-2-imidazoline (Compound (B)) was added into the mixture solution in batches, for thermal polymerization at 100° C. for 15 hours, so as to obtain a meta-stable state nitrogen-containing polymer of Example 2. The molar ratio of 5% 4,4′-diphenylmethane bismaleimide to 2,4-dimethyl-2-imidazoline was 2:1.

The meta-stable state nitrogen-containing polymer of Example 2 was a narrow polydispersity polymer having a GPC peak time of 22.4 min and a PDI of 1.2, as shown in FIG. 2. Furthermore, the meta-stable state nitrogen-containing polymer of Example 2 was re-induced to react at a temperature of 180° C., to convert the monomer into the polymer completely.

### EXAMPLE 3

Firstly, oligomer of phenylmethane maleimide (Compound (A)) was dissolved in NMP in an amount of 3%, to form a mixture solution. Next, 2,4-dimethyl-2-imidazoline (Compound (B)) was added into the mixture solution in batches, for thermal polymerization at 150° C. for 3 hours, so as to obtain a meta-stable state nitrogen-containing polymer of Example 3. The molar ratio of 3% oligomer of phenylmethane maleimide to 2,4-dimethyl-2-imidazoline was 4:1.

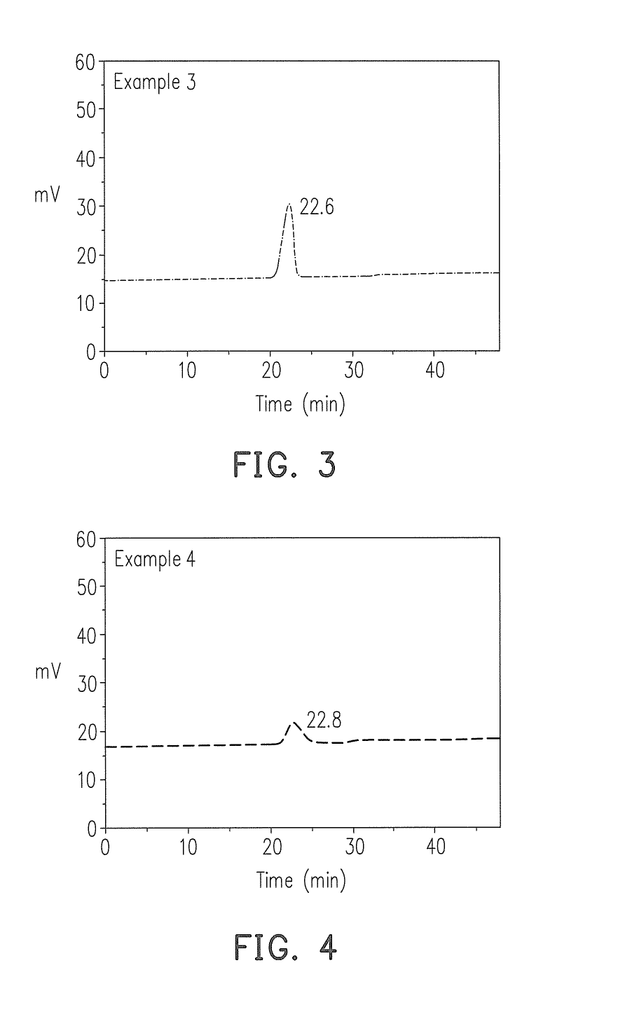

The meta-stable state nitrogen-containing polymer of Example 3 was a narrow polydispersity polymer having a GPC peak time of 22.6 min and a PDI of 1.2, as shown in FIG. 3. Furthermore, the meta-stable state nitrogen-containing polymer of Example 3 was re-induced to react at a temperature of 186° C., to convert the monomer into the polymer completely.

### EXAMPLE 4

Firstly, 4,4′-diphenylmethane bismaleimide (Compound (A)) was dissolved in NMP in an amount of 3%, to form a mixture solution. Next, imidazole (Compound (B)) was added into the mixture solution in batches, for thermal polymerization at 130° C. for 8 hours, so as to obtain a meta-stable state nitrogen-containing polymer of Example 4. The molar ratio of 3% 4,4′-diphenylmethane bismaleimide to imidazole was 4:1.

The meta-stable state nitrogen-containing polymer of Example 4 was a narrow polydispersity polymer having a GPC peak time of 22.8 min and a PDI of 1.3, as shown in FIG. 4. Furthermore, the meta-stable state nitrogen-containing polymer of Example 4 was re-induced to react at a temperature of 200° C., to convert the monomer into the polymer completely.

### EXAMPLE 5

Firstly, 1,6′-bismaleimide-(2,2,4-trimethyl)hexane (Compound (A)) was dissolved in GBL in an amount of 3%, to form a mixture solution. Next, pyridazine (Compound (B)) was added into the mixture solution in batches, for thermal polymerization at 100° C. for 12 hours, so as to obtain a meta-stable state nitrogen-containing polymer of Example 5. The molar ratio of 3% 1,6′-bismaleimide-(2,2,4-trimethyl)hexane to pyridazine was 2:1.

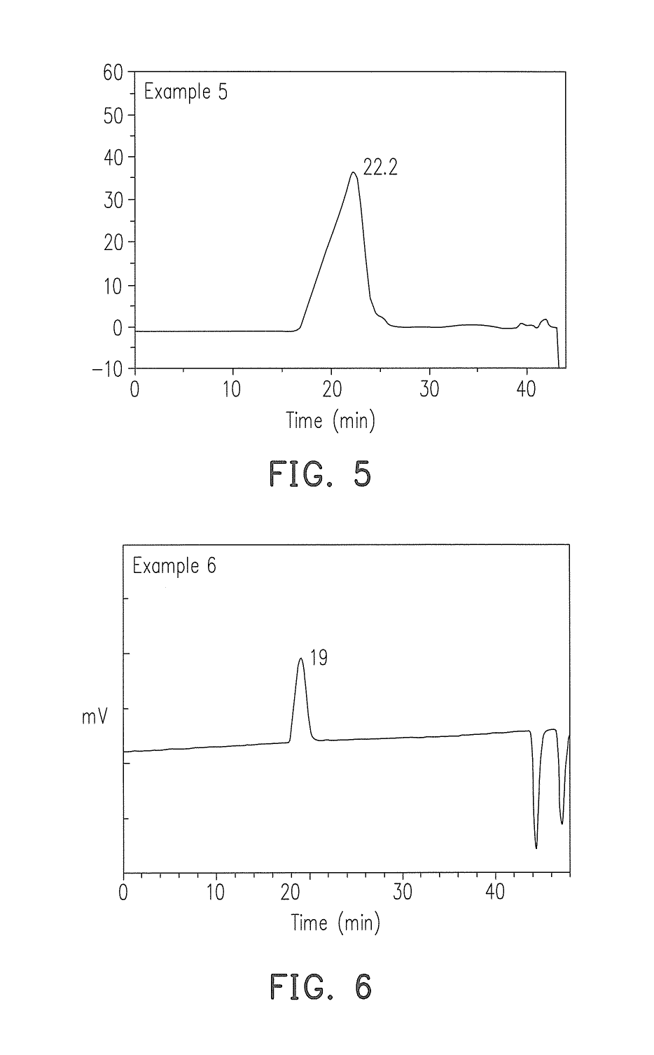

The meta-stable state nitrogen-containing polymer of Example 5 was a narrow polydispersity polymer having a GPC peak time of 22.2 min and a PDI of 1.5, as shown in FIG. 5. Furthermore, the meta-stable state nitrogen-containing polymer of Example 5 was re-induced to react at a temperature of 190° C., to convert the monomer into the polymer completely.

### EXAMPLE 6

Firstly, 2,2′-bis[4-(4-maleimidophenoxy)phenyl]propane (Compound (A)) was dissolved in GBL in an amount of 3%, to form a mixture solution. Next, pyridine (Compound (B)) was added into the mixture solution in batches, for thermal polymerization at 60° C. for 24 hours, so as to obtain a meta-stable state nitrogen-containing polymer of Example 6. The molar ratio of 3% 2,2′-bis[4-(4-maleimidophenoxy)phenyl]propane to pyridine was 4:1.

The meta-stable state nitrogen-containing polymer of Example 6 was a narrow polydispersity polymer having a GPC peak time of 19 min and a PDI of 1.2, as shown in FIG. 6. Furthermore, the meta-stable state nitrogen-containing polymer of Example 6 was re-induced to react at a temperature of 180° C., to convert the monomer into the polymer completely.

### EXAMPLE 7

Firstly, oligomer of phenylmethane maleimide (Compound (A)) was dissolved in EC/PC in an amount of 5%, to form a mixture solution. Next, 2,4,6-triamino-1,3,5,-triazine (Compound (B)) was added into the mixture solution in batches, for thermal polymerization at 130° C. for 12 hours, so as to obtain a meta-stable state nitrogen-containing polymer of Example 7. The molar ratio of 5% oligomer of phenylmethane maleimide to 2,4,6-triamino-1,3,5,-triazine was 2:1.

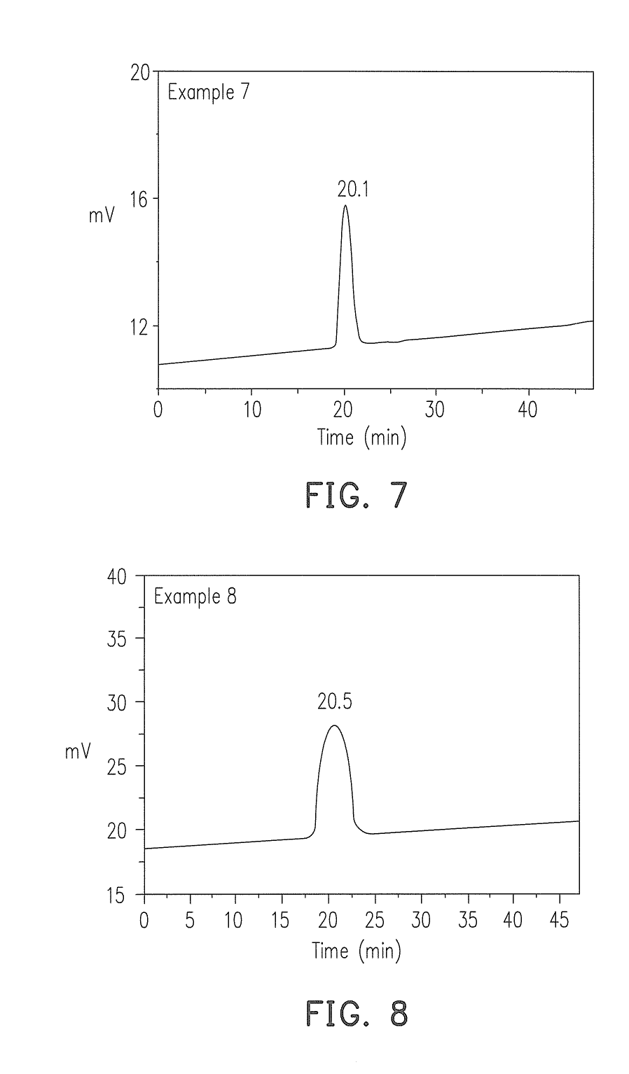

The meta-stable state nitrogen-containing polymer of Example 7 was a narrow polydispersity polymer having a GPC peak time of 20.1 min and a PDI of 1.1, as shown in FIG. 7. Furthermore, the meta-stable state nitrogen-containing polymer of Example 7 was re-induced to react at a temperature of 190° C., to convert the monomer into the polymer completely.

### EXAMPLE 8

Firstly, oligomer of phenylmethane maleimide (Compound (A)) was dissolved in GBL in an amount of 5%, to form a mixture solution. Next, 2,4-dimethyl-2-imidazoline (Compound (B)) was added into the mixture solution in batches, for thermal polymerization at 60° C. for 24 hours, so as to obtain a meta-stable state nitrogen-containing polymer of Example 8. The molar ratio of 5% oligomer of phenylmethane maleimide to 2,4-dimethyl-2-imidazoline was 10:1.

The meta-stable state nitrogen-containing polymer of Example 8 was a narrow polydispersity polymer having a GPC peak time of 20.5 min and a PDI of 1.5, as shown in FIG. 8. Furthermore, the meta-stable state nitrogen-containing polymer of Example 8 was re-induced to react at a temperature of 170° C., to convert the monomer into the polymer completely.

### EXAMPLE 9

Firstly, 2,2′-bis[4-(4-maleimidophenoxy)phenyl]propane (Compound (A)) was dissolved in GBL in an amount of 5%, to form a mixture solution. Next, 4-tert-butylpyridine (Compound (B)) was added into the mixture solution in batches, for thermal polymerization at 60° C. for 24 hours, so as to obtain a meta-stable state nitrogen-containing polymer of Example 9. The molar ratio of 5% 2,2′-bis[4-(4-maleimidophenoxy)phenyl]propane to 4-tert-butylpyridine was 4:1.

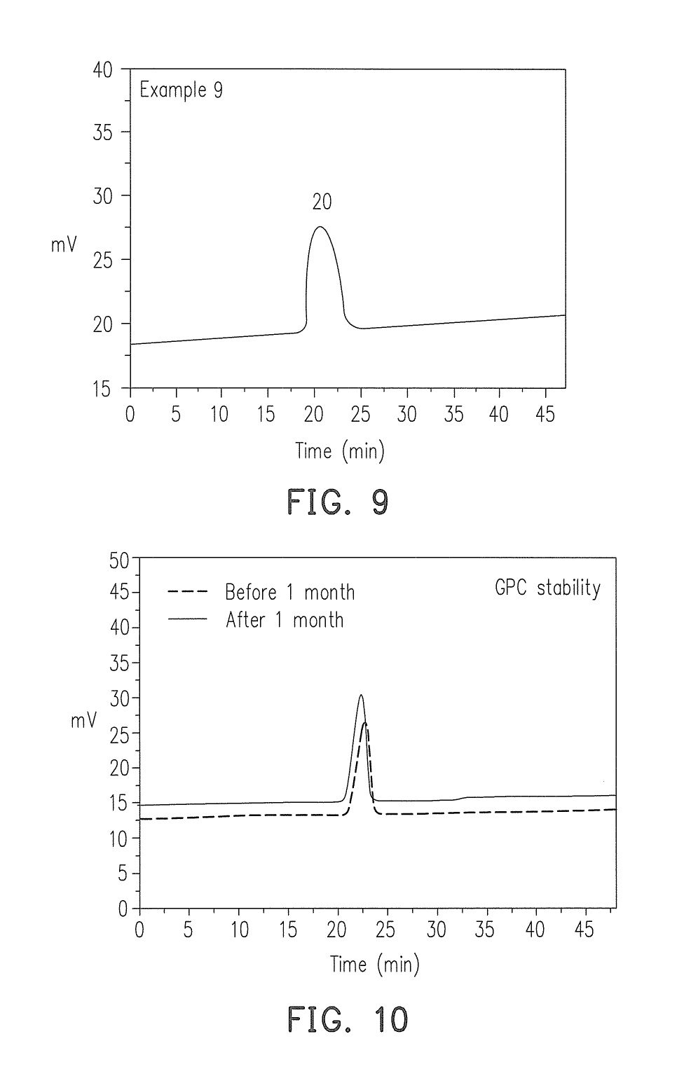

The meta-stable state nitrogen-containing polymer of Example 9 was a narrow polydispersity polymer having a GPC peak time of 20 min and a PDI of 1.5, as shown in FIG. 9. Furthermore, the meta-stable state nitrogen-containing polymer of Example 9 was re-induced to react at a temperature of 120° C., to convert the monomer into the polymer completely.

Table 3 summaries synthesis conditions and experimental results of Examples 1-9.

TABLE 3

| | | | Reaction | GPC peak | re-inducing |
|---|---|---|---|---|---|
| Example | Compound (A)/Compound (B) (molar ratio) | Solvent | conditions | time (min) | temperature |
| 1 | 3% oligomer of phenylmethane maleimide/ | EC/PC | 130° C., | 20.5 | 186° C. |
| | 2,4-dimethyl-2-imidazoline (2:1) | | 8 h | | |
| 2 | 5% 4,4′-diphenylmethane bismaleimide/ | GBL | 100° C., | 22.4 | 180° C. |
| | 2,4-dimethyl-2-imidazoline (2:1) | | 15 h | | |
| 3 | 3% oligomer of phenylmethane | NMP | 150° C., | 22.6 | 186° C. |
| | maleimide/2,4-dimethyl-2-imidazoline (4:1) | | 3 h | | |
| 4 | 3% 4,4′-diphenylmethane bismaleimide/ | NMP | 130° C., | 22.8 | 200° C. |
| | imidazole (4:1) | | 8 h | | |
| 5 | 3% 1,6′-bismaleimide-(2,2,4-trimethyl)hexane/ | GBL | 100° C., | 22.2 | 190° C. |
| | pyridazine (2:1) | | 12 h | | |
| 6 | 3% 2,2′-bis[4-(4-maleimidophenoxy)phenyl]propane/ | GBL | 60° C., | 19 | 180° C. |
| | pyridine (4:1) | | 24 h | | |
| 7 | 5% oligomer of phenylmethane maleimide/ | EC/PC | 130° C., | 20.1 | 190° C. |
| | 2,4,6-triamino-1,3,5,-triazine (2:1) | | 12 h | | |
| 8 | 5% oligomer of phenylmethane maleimide/ | EC/PC | 80° C., | 20.5 | 170° C. |
| | 2,4-dimethyl-2-imidazoline (10:1) | | 18 h | | |
| 9 | 5% 2,2′-bis[4-(4-maleimidophenoxy)phenyl]propane/ | GBL | 60° C., | 20 | 120° C. |
| | 4-tert-butylpyridine (4:1) | | 24 h | | |

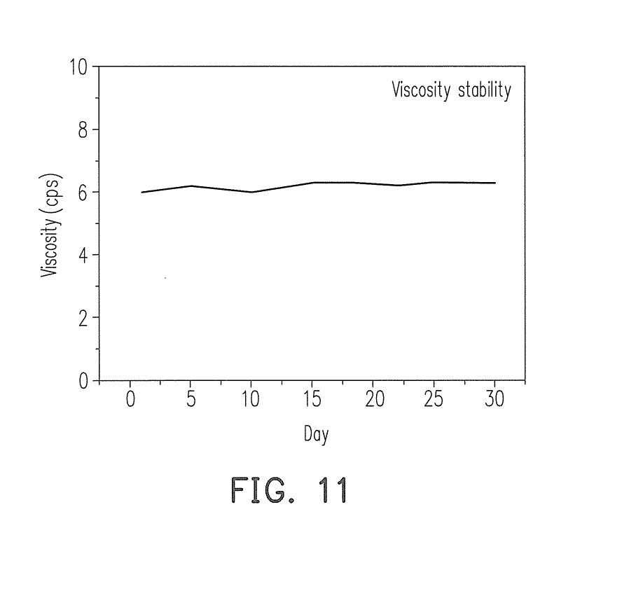

Furthermore, GPC stability test and viscosity stability test were also performed on the meta-stable state nitrogen-containing polymer of Example 3, as shown in FIGS. 10-11. Referring to FIG. 10, the variance of particle size of the meta-stable state nitrogen-containing polymer of Example 3 was lower than 2% after being stored at 55° C. for 1 month. Referring to FIG. 11, the variance of viscosity of the meta-stable state nitrogen-containing polymer of Example 3 was lower than 2% after being stored at 55° C. for 1 month.

In the above embodiments, Compound (B) is described with a heterocyclic amino aromatic derivative as a nucleophilic initiator as an example; however, the disclosure is not limited thereto. Persons of ordinary skill in the art should appreciate that, Compound (B) may also be a tertiary amine or a secondary amine, which is reacted with Compound (A) (that is, a monomer with a reactive terminal functional group), to generate a meta-stable state nitrogen-containing polymer.

In summary, the meta-stable state nitrogen-containing polymer of the disclosure has the advantages of both “two-liquid type” and “one-liquid type” polymers but without the drawbacks of the same. In details, the meta-stable state nitrogen-containing polymer of the disclosure does not require a mixing step and can be stored at room temperature (or above room temperature) for a long period of time, and the viscosity thereof will not change drastically after unsealing.

Furthermore, the meta-stable state nitrogen-containing polymer of the disclosure has part of the functional groups remained, which is beneficial to the subsequent processing, and optionally, the unreacted function groups may be facilitated to react by heating or applying a voltage. The application of the meta-stable state nitrogen-containing polymer of the disclosure is wide, such as an electrolyte additive of a secondary battery, a water-keeping layer of a fuel cell, a solid electrolyte, etc.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A meta-stable state nitrogen-containing polymer, being formed by reacting Compound (A) and Compound (B), wherein Compound (A) is a monomer with a reactive terminal functional group, Compound (B) is a heterocyclic amino aromatic derivative as an initiator, and a molar ratio of Compound (A) to Compound (B) is from 10:1 to 1:10.

2. The meta-stable state nitrogen-containing polymer according to claim 1, wherein Compound (B) is represented by one of Formula (1) to Formula (9): 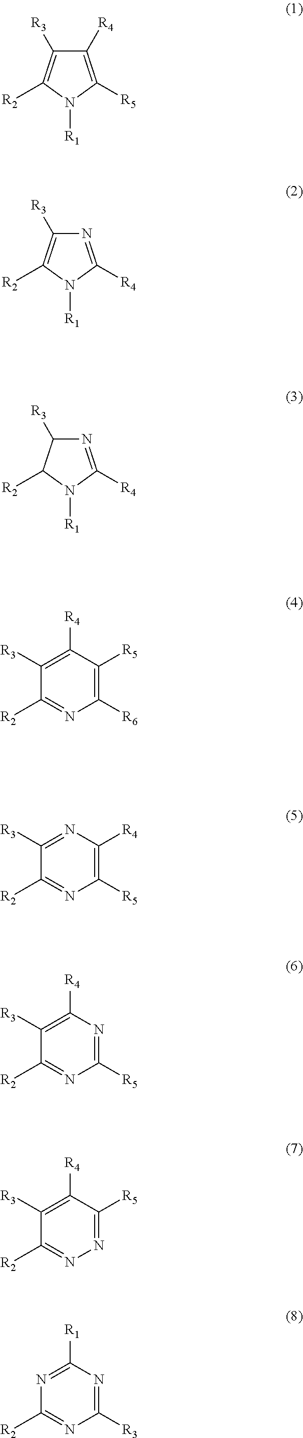 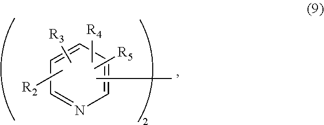
wherein R₁ is hydrogen, alkyl, alkenyl, phenyl, dimethylamino, or —NH₂; and R₂, R₃, R₄, and R₅ are each independently hydrogen, alkyl, alkenyl, halo, or —NH₂.

3. The meta-stable state nitrogen-containing polymer according to claim 2, wherein Compound (B) comprises imidazole, an imidazole derivative, pyrrole, an pyrrole derivative, pyridine, 4-tert-butylpyridine, 3-butylpyridine, 4-dimethylaminopyridine, 2,4,6-triamino-1,3,5,-triazine, 2,4-dimethyl-2-imidazoline (D242), pyridazine, pyrimidine, or pyrazine.

4. The meta-stable state nitrogen-containing polymer according to claim 1, wherein Compound (A) comprises a maleimide, poly(ethylene glycol) dimethacrylate, bis[[4-[(vinyloxy)methyl]cyclohexyl]methyl]isophthalate), or triallyl trimellitate,
wherein the maleimide is represented by one of Formula (10) to Formula (13): 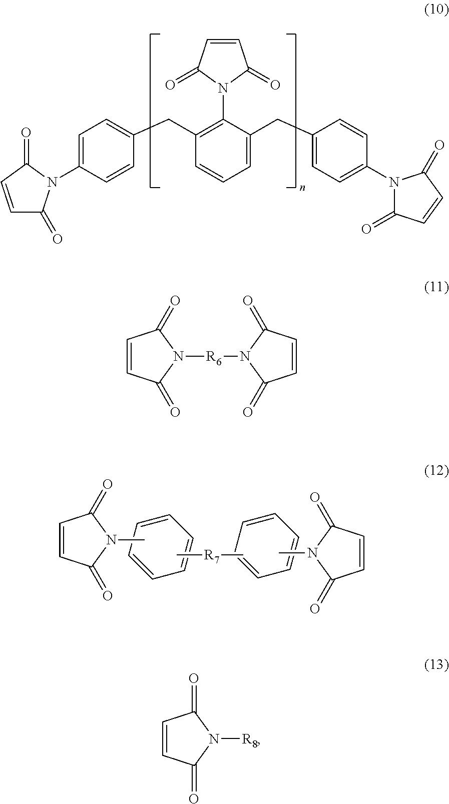
wherein n is an integer of 0 to 4; R₆ is —RCH₂R′—, —RNH₂R—, —C(O)CH₂—, —R′OR″OR′—, —CH₂OCH₂—, —C(O)—, —O—, —O—O—, —S—, —S—S—, —S(O)—, —CH₂S(O)CH₂—, —(O)S(O)—, —C₆H₅—, —CH₂(C₆H₅)CH₂—, —CH₂(C₆H₅)(O)—, phenylene, biphenylene, substituted phenylene, or substituted biphenylene, R is hydrogen or C₁₋₄ alkyl, R′ is C₁₋₄ alkyl, and R″ is C₁₋₄ alkyl; R₇ is —RCH₂—, —C(O)—, —C(CH₃)₂—, —O—, —O—O—, —S—, —S—S—, —(O)S(O)—, or —S(O)—; and R₈ is hydrogen, C₁₋₄ alkyl, phenyl, benzyl, cyclohexyl, or N-methoxy carbonyl.

5. The meta-stable state nitrogen-containing polymer according to claim 4, wherein the maleimide comprises 4,4′-diphenylmethane bismaleimide, an oligomer of phenylmethane maleimide, m-phenylene bismaleimide, 2,2′-bis[4-(4-maleimidophenoxy)phenyl]propane, 3,3′-dimethyl-5,5′-diethyl-4,4′-diphenylmethane bismaleimide, 4-methyl-1,3-phenylene maleimide, 1,6′-bismaleimide-(2,2,4-trimethyl)hexane, 4,4′-diphenylether bismaleimide, 4,4′-diphenylsulfone bismaleimide, 1,3-bis(3-maleimidophenoxy)benzene, or 1,3-bis(4-maleimidophenoxy)benzene.

6. The meta-stable state nitrogen-containing polymer according to claim 1, wherein the molar ratio of Compound (A) to Compound (B) is from 1:1 to 5:1.

7. The meta-stable state nitrogen-containing polymer according to claim 1, wherein the meta-stable state nitrogen-containing polymer is a narrow polydispersity polymer.

8. The meta-stable state nitrogen-containing polymer according to claim 1, wherein the meta-stable state nitrogen-containing polymer is re-induced to react at a temperature of 120-200° C., to convert the meta-stable state nitrogen-containing polymer into a macromolecular polymer completely.
